SEQUENCE LISTING (52 sequences)

SEQ ID NO: 1 - RNA, 45 nt, synthetic construct
  source 1..45: mol_type = other RNA
aagtcatggctagtcatgctagccagttcgagcaatcgactgcta

SEQ ID NO: 2 - RNA, 28 nt, synthetic construct
  source 1..28: mol_type = other RNA
cgattgctcgaactggctagcatgacta

SEQ ID NO: 3 - RNA, 28 nt, synthetic construct
  source 1..28: mol_type = other RNA
cgtgagagtaattctgragcacttagca

SEQ ID NO: 4 - RNA, 28 nt, synthetic construct
  source 1..28: mol_type = other RNA
gtaattctgragcacttagcaargcytt

SEQ ID NO: 5 - RNA, 28 nt, synthetic construct
  source 1..28: mol_type = other RNA
tgragcacttagcaargcyttrtcycta

SEQ ID NO: 6 - RNA, 28 nt, synthetic construct
  source 1..28: mol_type = other RNA
cttagcaargcyttrtcyctaacyaayt

SEQ ID NO: 7 - RNA, 28 nt, synthetic construct
  source 1..28: mol_type = other RNA
argcyttrtcyctaacyaaytgyacyac

SEQ ID NO: 8 - RNA, 28 nt, synthetic construct
  source 1..28: mol_type = other RNA
rtcyctaacyaaytgyacyacagcaatg

SEQ ID NO: 9 - RNA, 28 nt, synthetic construct
  source 1..28: mol_type = other RNA
acyaaytgyacyacagcaatgttaaara

SEQ ID NO: 10 - RNA, 28 nt, synthetic construct
  source 1..28: mol_type = other RNA
gyacyacagcaatgttaaaraayttgtg

SEQ ID NO: 11 - RNA, 28 nt, synthetic construct
  source 1..28: mol_type = other RNA
agcaatgttaaaraayttgtgtttytay

SEQ ID NO: 12 - RNA, 28 nt, synthetic construct
  source 1..28: mol_type = other RNA
ttaaaraayttgtgtttytayagycagg

SEQ ID NO: 13 - RNA, 28 nt, synthetic construct
  source 1..28: mol_type = other RNA
ayttgtgtttytayagycaggartcrcc

SEQ ID NO: 14 - RNA, 28 nt, synthetic construct
  source 1..28: mol_type = other RNA
tttytayagycaggartcrcctcartct

SEQ ID NO: 15 - RNA, 28 nt, synthetic construct
  source 1..28: mol_type = other RNA
agycaggartcrcctcartcttayaatt

SEQ ID NO: 16 - RNA, 28 nt, synthetic construct
  source 1..28: mol_type = other RNA
artcrcctcartcttayaattcaactgg

SEQ ID NO: 17 - RNA, 28 nt, synthetic construct
  source 1..28: mol_type = other RNA
tcartcttayaattcaactggycctgay

SEQ ID NO: 18 - RNA, 28 nt, synthetic construct
  source 1..28: mol_type = other RNA
tayaattcaactggycctgayacyggta

SEQ ID NO: 19 - RNA, 28 nt, synthetic construct
  source 1..28: mol_type = other RNA
caactggycctgayacyggtagaytraa

SEQ ID NO: 20 - RNA, 28 nt, synthetic construct
  source 1..28: mol_type = other RNA
ycctgayacyggtagaytraaattytct

SEQ ID NO: 21 - RNA, 28 nt, synthetic construct
  source 1..28: mol_type = other RNA
acyggtagaytraaattytctttrtcyt

SEQ ID NO: 22 - RNA, 28 nt, synthetic construct
  source 1..28: mol_type = other RNA
gaytraaattytctttrtcytacaagga

SEQ ID NO: 23 - RNA, 28 nt, synthetic construct
  source 1..28: mol_type = other RNA
attytctttrtcytacaaggaacargtr

SEQ ID NO: 24 - RNA, 28 nt, synthetic construct
  source 1..28: mol_type = other RNA
ttrtcytacaaggaacargtrggrggya

SEQ ID NO: 25 - RNA, 28 nt, synthetic construct
  source 1..28: mol_type = other RNA
acaaggaacargtrggrggyaayagaga

SEQ ID NO: 26 - RNA, 28 nt, synthetic construct
  source 1..28: mol_type = other RNA
acargtrggrggyaayagagagttgtac

SEQ ID NO: 27 - RNA, 28 nt, synthetic construct
  source 1..28: mol_type = other RNA
ggrggyaayagagagttgtacatyggrg

SEQ ID NO: 28 - RNA, 28 nt, synthetic construct
  source 1..28: mol_type = other RNA
ayagagagttgtacatyggrgayytrag

SEQ ID NO: 29 - RNA, 28 nt, synthetic construct
  source 1..28: mol_type = other RNA
gttgtacatyggrgayytragracraag

SEQ ID NO: 30 - RNA, 28 nt, synthetic construct
  source 1..28: mol_type = other RNA
atyggrgayytragracraagatgttya

SEQ ID NO: 31 - RNA, 28 nt, synthetic construct
  source 1..28: mol_type = other RNA
ayytragracraagatgttyacraggct

SEQ ID NO: 32 - RNA, 28 nt, synthetic construct
  source 1..28: mol_type = other RNA
racraagatgttyacraggctcatygar

SEQ ID NO: 33 - RNA, 28 nt, synthetic construct
  source 1..28: mol_type = other RNA
atgttyacraggctcatygargattact

SEQ ID NO: 34 - RNA, 28 nt, synthetic construct
  source 1..28: mol_type = other RNA
craggctcatygargattactttgaagc

SEQ ID NO: 35 - RNA, 28 nt, synthetic construct
  source 1..28: mol_type = other RNA
catygargattactttgaagcrctyaca

SEQ ID NO: 36 - RNA, 28 nt, synthetic construct
  source 1..28: mol_type = other RNA
gattactttgaagcrctyacatcacaat

SEQ ID NO: 37 - RNA, 28 nt, synthetic construct
  source 1..28: mol_type = other RNA
ttgaagcrctyacatcacaattrrcrgg

SEQ ID NO: 38 - RNA, 28 nt, synthetic construct
  source 1..28: mol_type = other RNA
rctyacatcacaattrrcrggcagytgy

SEQ ID NO: 39 - RNA, 28 nt, synthetic construct
  source 1..28: mol_type = other RNA
tcacaattrrcrggcagytgyytaaaca

SEQ ID NO: 40 - RNA, 28 nt, synthetic construct
  source 1..28: mol_type = other RNA
trrcrggcagytgyytaaacaatgagaa

SEQ ID NO: 41 - RNA, 28 nt, synthetic construct
  source 1..28: mol_type = other RNA
cagytgyytaaacaatgagaargartty

SEQ ID NO: 42 - RNA, 28 nt, synthetic construct
  source 1..28: mol_type = other RNA
ytaaacaatgagaargarttygacaatg

SEQ ID NO: 43 - RNA, 28 nt, synthetic construct
  source 1..28: mol_type = other RNA
atgagaargarttygacaatgccatytt

SEQ ID NO: 44 - RNA, 28 nt, synthetic construct
  source 1..28: mol_type = other RNA
rgarttygacaatgccatyttrtcaatg

SEQ ID NO: 45 - RNA, 28 nt, synthetic construct
  source 1..28: mol_type = other RNA
gacaatgccatyttrtcaatgaarytra

SEQ ID NO: 46 - RNA, 28 nt, synthetic construct
  source 1..28: mol_type = other RNA
ccatyttrtcaatgaarytraatgtctc

SEQ ID NO: 47 - RNA, 28 nt, synthetic construct
  source 1..28: mol_type = other RNA
rtcaatgaarytraatgtctcatcrgca

SEQ ID NO: 48 - RNA, 28 nt, synthetic construct
  source 1..28: mol_type = other RNA
aarytraatgtctcatcrgcacaygtat

SEQ ID NO: 49 - RNA, 28 nt, synthetic construct
  source 1..28: mol_type = other RNA
atgtctcatcrgcacaygtatcatayag

SEQ ID NO: 50 - RNA, 28 nt, synthetic construct
  source 1..28: mol_type = other RNA
atcrgcacaygtatcatayagyatggat

SEQ ID NO: 51 - RNA, 28 nt, synthetic construct
  source 1..28: mol_type = other RNA
caygtatcatayagyatggatcacagya

SEQ ID NO: 52 - RNA, 28 nt, synthetic construct
  source 1..28: mol_type = other RNA
catayagyatggatcacagyaagtgggg

(19) United States
(12) Patent Application Publication
(10) Pub. No.: US 2024/0254571 A1
(43) Pub. Date: Aug. 1, 2024

(54) Degenerate CRISPR Cas13a crRNAs for Detection of Highly Variable RNA Sequences

(71) Applicant: The Government of the United States of America, as represented by the Secretary of the Navy, Arlington, VA (US)

(72) Inventors: Tomasz A. Leski, Falls Church, VA (US); Joseph R. Spangler, La Plata, MD (US); Zheng Wang, Burke, VA (US); Scott N. Dean, Bowie, MD (US); David A. Stenger, Annapolis, MD (US)

(21) Appl. No.: 18/422,206

(22) Filed: Jan. 25, 2024

Related U.S. Application Data
(60) Provisional application No. 63/441,196, filed on Jan. 26, 2023.

(51) Int. Cl. C12Q 1/70; C12N 9/22; C12N 15/11; G16B 40/00

(52) U.S. Cl. CPC C12Q 1/701; C12N 9/22; C12N 15/11; G16B 40/00; C12N 2310/20; C12N 2320/10

(57) ABSTRACT

A technique for the design of minimum CRISPR RNA (crRNA) sets aids in the detection of diverse nucleic acid targets using sequence degeneracy. As a working example, candidate degenerate Cas13a crRNA sets were designed for detection of diverse RNA targets (Lassa virus). A decision tree machine learning (ML) algorithm (RuleFit) was applied to define the top attributes that determine the specificity of degenerate crRNAs to elicit collateral nuclease activity. This general ML approach can be applied to the design of degenerate crRNA sets for any CRISPR/Cas system.

### CROSS-REFERENCE TO RELATED APPLICATIONS

This application claims the benefit of U.S. Provisional Patent Application No. 63/441,196 filed on Jan. 26, 2023, the entirety of which, inclusive of appendices, is incorporated herein by reference.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 211390.

### INCORPORATION BY REFERENCE

This Application incorporates by reference the Sequence Listing XML file submitted herewith via the patent office electronic filing system having the file name “211390 sequences.xml” and created on Jan. 24, 2024 with a file size of 47,195 bytes.

### BACKGROUND

Rapid and specific detection of infectious agents is necessary for the efficient treatment of their corresponding diseases and for epidemiologic surveillance. Nucleic-acid-based detection methods such as polymerase chain reaction (PCR) could be considered a gold standard for infectious disease diagnostics, offering high specificity and sensitivity, but are limited by high cost, the requirement of expensive instrumentation, and need for highly trained personnel. Clustered Regularly Interspaced Short Palindromic Repeat (CRISPR)-based detection methods and CRISPR-Associated Protein (CAS) systems emerged recently as an alternative to PCR-based diagnostics with the potential to develop less complex detection assays but offering the diagnostic accuracy of PCR.

The enzymes and methods referred to as CRISPR tools have been in the recent years exploited to carry out a number of functions, from DNA and RNA mutagenesis, modification, and labeling to enrichment, detection, and characterization of known and unknown nucleic acid (NA) sequences. The Cas proteins, which, in their most important function, are essentially just programmable, RNA-guided, DNA or RNA binding proteins, can be applied to direct a number of actions that one might wish to perform on a NA target.

For the CRISPR-based detection of nucleic acid targets (especially those with high variability), it is important to understand the relationship between the sequence and function of the CRISPR guide RNA (crRNA) molecules defining the specificity for the target NA. CRISPR detection applications rely on “design rules” for crRNAs against specific DNA (DETECTR) or RNA (SHERLOCK) targets using Cas12 and Cas13, respectively. Empirical design rules for RNA targets using Cas13 were originally based on observed experimental results of collateral cleavage of fluorescent molecular beacons following specific activation of Cas13 using a relatively small number of candidate crRNAs. Another approach for automated, target-specific crRNA design for large numbers of different pathogens uses neural network-based machine learning algorithms trained on data obtained from massively parallel Cas13a crRNA collateral nuclease screens. Such an approach, however, depends on the selection of multiple specific guide RNAs to cover a broad phylogenetic target range.

A need exists for a technique involving for the detection of highly variable nucleic acid targets and where the design rules for degenerate crRNAs have application to a wide array of targets.

### BRIEF SUMMARY

Described herein is a technique to identify the smallest number (or a close approximation thereto) of degenerate crRNAs that could activate Cas13a collateral activity to produce a simple binary result for the presence or absence of any member of a phylogenetically diverse group.

In one embodiment, a method for detecting members of a phylogenetically diverse group includes (1) identifying conserved regions in a set of diverse nucleic acids in the phylogenetically diverse group; (2) designing candidate degenerate complementary spacer regions of CRISPR guide RNAs (crRNAs) corresponding to the conserved regions; (3) conducting high-throughput screening of the candidate degenerate crRNAs against complementary synthetic targets to obtain high performing degenerate crRNAs; (4) conducting high-throughput screening of the high performing degenerate crRNAs against targets representing at least a majority of the phylogenetically diverse group and at least one target representing a near neighbor to the phylogenetically diverse group to obtain a dataset; and (5) using a machine learning algorithm to analyze the dataset to identify generalizable crRNA design rules for detection of members of the phylogenetically diverse group.

A further embodiments include a CRISPR guide RNA (crRNA) comprising a nucleic acid sequence identified as described herein, as well as Cas-based assay systems incorporating such crRNAs.

### BRIEF DESCRIPTION OF THE DRAWINGS

FIG. 1 depicts the design of the crRNAs targeting conserved regions of Lassa virus (LASV) L and GPC genes (representing “large,” and “glycoprotein complex,” respectively) to provide an overview of the LASV crRNA design strategy. Conserved regions of the L (370 bp) and GPC (211 bp or 283 bp) genes were selected and consensus sequences for lineage II or lineage IV were constructed. The degenerate spacer sequences of the crRNAs were designed by tiling the 28nt sequences across the consensus target sequences.

FIGS. 2A through 2D show performance testing results for crRNAs designed for GPC and L targets. The height of the bars reflect the cumulative background subtracted fluorescence signal (RFU) obtained for each of the tested crRNAs with their corresponding targets. The graphs of FIGS. 2A and 2B show the results for GPC (FIG. 2A) and L (FIG. 2B) targets designed based on LASV lineage II sequences. The graphs of FIGS. 2C and 2D show the results for GPC and L targets, respectively, designed based on LASV lineage IV sequences. The numbers inside the bars and the intensity of their shading reflect degeneracy of the spacer for each of the tested crRNA molecules. The vertical lines denote the signal thresholds used to classify the crRNA performance into low (signal less than 20% of the maximum signal), medium (signal 20% or higher but less than 80%) and high (signal equal or higher than 80%).

FIGS. 3A to 3C illustrate the taxonomic relationship of LASV and near-neighbor Old World Arenaviruses used to design target sequences for the working example. The Arenaviridae family taxonomic tree was reproduced from the report of International Committee on Taxonomy of Viruses (ICTV). The branch of the tree enclosed in box seen in FIGS. 3B and 3C includes the Old World Arenavirus (OWA) group. LASV is seen near the bottom of FIG. 3B and the near-neighbor species used to design the target sequences are enclosed in boxes. Dandenong virus (closely related to LCMV) and Morogoro (closely related to Mopeia virus) are not shown.

FIGS. 4A and 4B show crRNA #5 performance versus LASV lineages and near neighbors with results of testing of crRNAs #5_LII and #5_LIV against a panel of 12 LASV targets representing all currently known lineages (I-VII) and 11 near-neighbor old world arenavirus (OWA) targets. The height of the bars reflect the cumulative background subtracted fluorescence obtained for each of the tested crRNAs against the specific target.

FIG. 5 depicts the structure of an exemplary Cas13a crRNA and target with the following features: protospacer (target) sequence top, SEQ ID NO: 1 representing the part of the target complementary to the spacer sequence; protospacer flanking sites (PFS) boxed with numbers 1 and 2; crRNA spacer with SEQ ID NO: 2 numbered (1-28) in 5′ to 3′ direction (note this is right to left in the image); and shaded hairpin/constant part of the crRNA. Location of features defined in RuleFit model are indicated below the spacer sequence.

FIGS. 6A and 6B illustrate RuleFit model performance and the identified most significant features for Watson-Crick base pairing. FIG. 6A has a confusion matrix showing percentages of actual assay outcomes versus outcomes produced by the classifier model. FIG. 6B show the relative effect of features on model predictions: n-number of mismatches, IQR—interquartile range for mismatch positions (characterizes distribution of mismatches with IRQ=14 indicating even distribution and IRQ>14 and IRQ<14 indicating clustering), min—position of mismatch closest to 5′ end of crRNA, PFS_1—nucleotide at protospacer flanking site #1, range—distance between the furthest apart mismatches, remaining features corresponding to various regions of crRNA as described in FIG. 5.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Overview

The approach to identify the smallest number (or a close approximation thereto) of degenerate crRNAs to detect members of a phylogenetically diverse group via Cas13a collateral activity used Lassa virus (LASV) as a model taxon. This entailed the following steps:
(1) selection of conserved regions of LASV genomes,
(2) tiled design of degenerate sequences in the complementary spacer regions of crRNAs across those conserved genomic regions,
(3) high-throughput screening of the candidate degenerate crRNAs against complementary synthetic targets,
(4) high-throughput screening of the selected high performing degenerate crRNAs against targets representing all known lineages of LASV (the intended target) and target representing closely related viral species (near-neighbors), and
(5) use of a machine learning algorithm to analyze the datasets obtained in step (4) to identify the generalizable crRNA design rules for detection of highly variable targets.

Additional details can be found in the Appendices of U.S. Provisional Patent Application No. 63/441,196.

Results demonstrated that a “single” degenerate crRNA, being a de-facto mixture of 512 distinct crRNAs with all permutations of bases in the degenerate sites of the spacer, can detect targets representing all seven currently identified LASV lineages. Detection was independent of the presence of a genomic nucleic acid background and only one closely-related near-neighbor cross-reacted out of eleven tested. The analysis of the experimental dataset revealed that the primary variable predicting greater than threshold collateral cleavage (positive detection) was a total number of mismatches between the crRNA spacer and the target region. To a lesser extent, the signal was affected by the positions and distribution of the mismatches and identity of nucleotides in the protospacer flanking sites (PFS). This indicates that many fewer crRNAs can be dedicated to the detection of phylogenetically diverse targets in assays having a limited number of test channels. The approach could also prove useful for other CRISPR-Cas applications.

The Lassa virus (LASV) was used to demonstrate this technique for the design and use of degenerate crRNAs for detection of highly diverse RNA targets. The LASV genome has conserved regions, however even these exhibit a significant degree of variability among the seven known lineages of the virus.

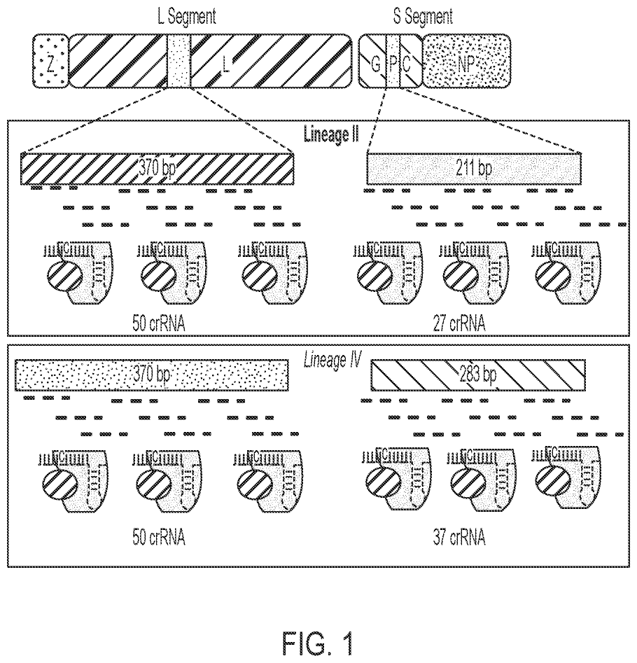

Multiple degenerate crRNA were designed for each of the conserved targets in L and GPC genes of lineage I and lineage IV LASV (FIG. 1). The crRNAs were synthesized using standard in vitro methods on templates of commercially obtained DNA oligonucleotides. The performance of these degenerate crRNAs was tested using Cas13a assay to identify the high-performing examples.

Both crRNA synthesis and Cas13a activity assays were conducted using a high throughput workflow in 384 well plates with fluid transfer handled by Echo 525 acoustic liquid handler (Beckman Coulter, Indianapolis, IN) using the Plate Reformat software provided by the manufacturer. In order to generate crRNAs, 27 to 50 crRNA transcription reactions were set up using reagent volumes as described above for an individual reaction. The reactions included the tested crRNAs and a negative control (crRNA template oligo replaced with TE buffer). First, master mix containing all reaction components except for template oligonucleotide were distributed using Echo instrument from 6 well Echo qualified reservoir plate to Echo qualified 384 well microplate. 23.5 μL of the master-mix were transferred to each well. Subsequently 1.5 μL of the crRNA template oligonucleotides were added to each well containing master-mix using Echo instrument from a previously prepared Echo qualified 384 well microplate. The plates were spun briefly in a centrifuge at approximately 1500 g to bring all the liquid to the bottom of the wells and remove air bubbles. The plate was sealed and incubated for 2 hours at 37° C. After incubation, the plates with transcribed crRNAs were stored in −80° C. To determine the efficacy of each crRNA, Cas13a nuclease activity assays were conducted using Cas13a enzyme from L. wadei which was synthesized and purified by GenScript Biotech (Piscataway, NJ). The enzyme was stored and diluted using the storage buffer (50 mM Tris-HCl, 600 mM NaCl, 5% Glycerol, 2 mM DTT, pH 7.5). Each nuclease activity assay was performed in 20 μL reaction that included 1 μL of 1 μM Cas13a, 1 μL of 2 μM RNase alert v.2 (from RNaseAlert™ QC System v2, ThermoFisher, Grand Island, NY), 17.2 μL of nuclease assay buffer (40 mM Tris-HCl, 60 mM NaCl, 6 mM MgCl₂, pH 7.3), 0.4 μL of crRNA (from unpurified transcription reaction) and 0.4 μL of target RNA (50 ng/μL for full length target or 12 ng/μL for short target). For each crRNA a total of six reactions were set up, with three target negative reactions and three target positive. First, master mix containing all reaction components except for crRNA and target RNA were distributed using Echo instrument from 6 well Echo qualified Reservoir plate to a 384 well assay plate (black with clear flat bottom, cat #3762, Corning Life Sciences, Tewksbury, MA). A total volume of 19.2 μL of the master-mix was transferred to each well. Next, 0.4 μL crRNAs from previously prepared 384 well microplate were transferred using Echo instrument to the wells containing the master-mix in such a way that each crRNA was added to 6 subsequent wells in the reaction plate. Finally, 0.4 μL of the target RNAs (previously placed in the area of the crRNA plate not occupied by transcribed crRNAs) were added to three of the wells for each crRNA. The Cas13a reaction plates were spun briefly in a centrifuge at approximately 1500×g to bring the liquid to the bottom of the wells and remove air bubbles. Immediately after spinning, the reaction plates were sealed using the MicroAmp sealers. The plates were incubated in Biotek Synergy Neo2 plate reader (Biotek, Winooski, VT) at 37° C. and fluorescence was read from the bottom of the wells every 5 minutes for 2 hours using excitation at 490 nm, emission at 520 nm and gain set at 100. The integrated background corrected final fluorescence values reflecting the Cas13a RNase activation for each of the crRNAs was calculated by subtracting the sum of averages of fluorescence measured for template negative samples over the course of the experiment (25 measurements) from sum of averages for template positive samples. The crRNAs were classified into three groups based on the integrated, background subtracted, fluorescent signal relative to the highest signal obtained. Thee performance classes were defined: high performance (with signal at 80% or higher), intermediate (signal lower than 80% but higher than 20%) and low (signal at 20% or lower).

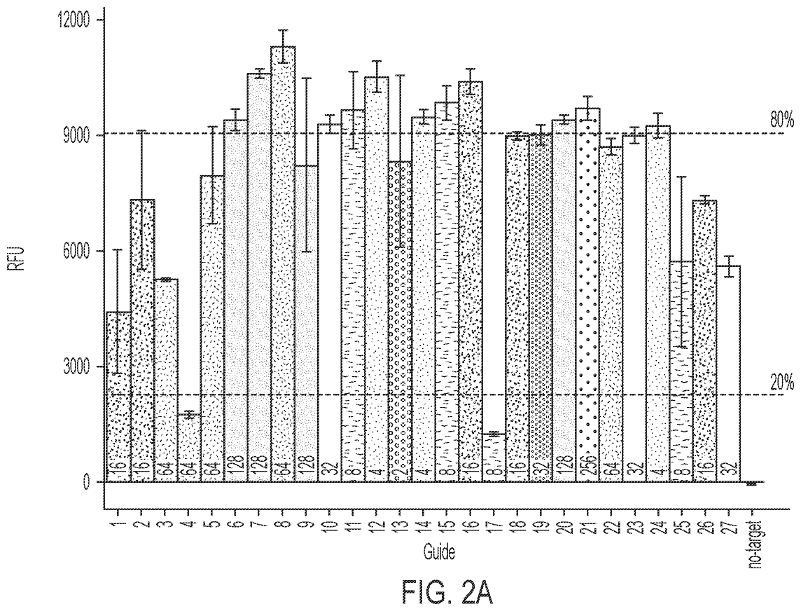

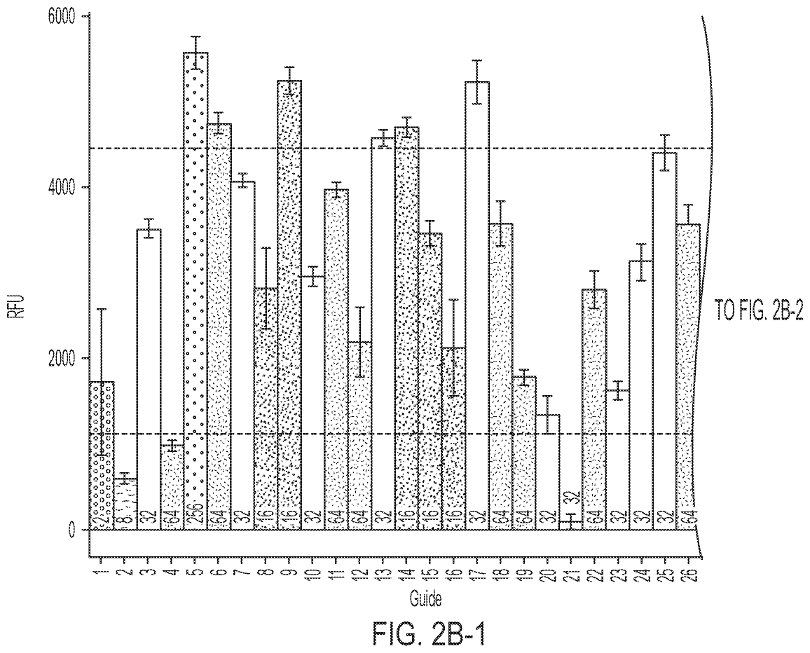

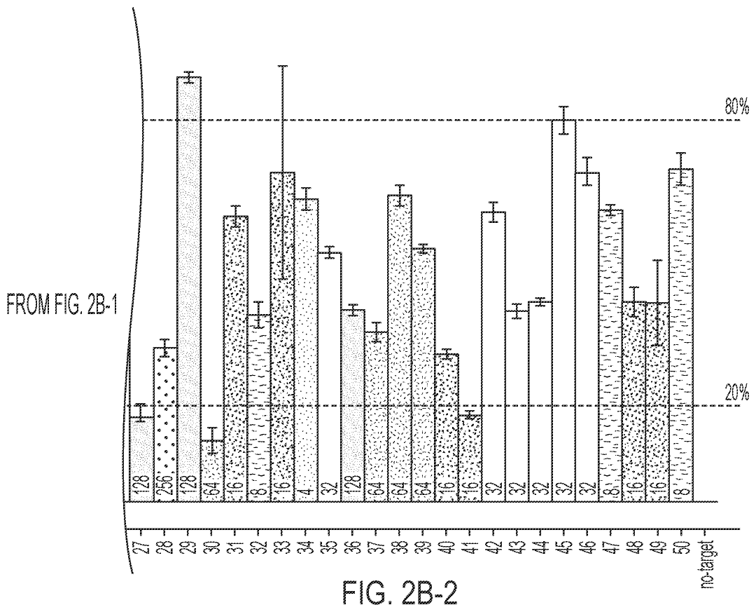

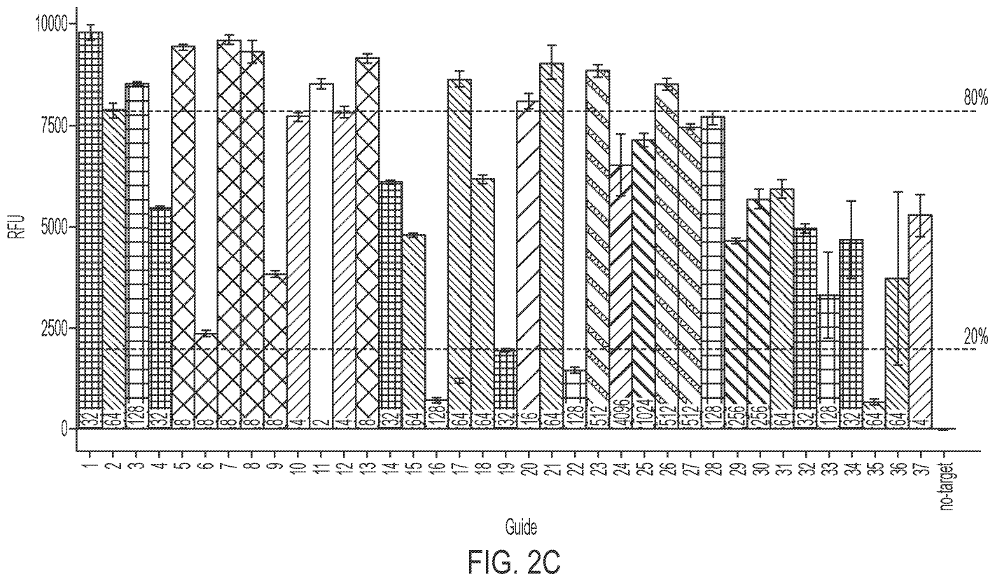

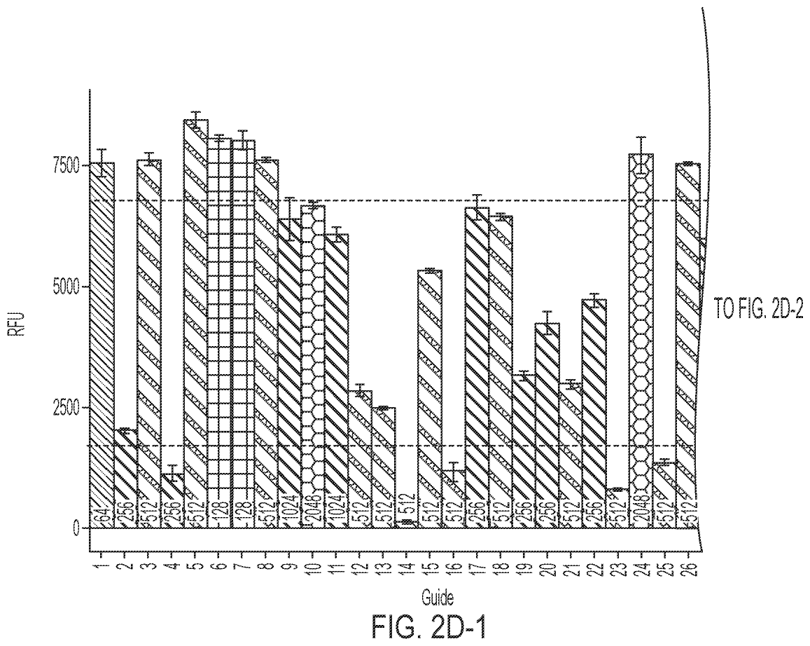

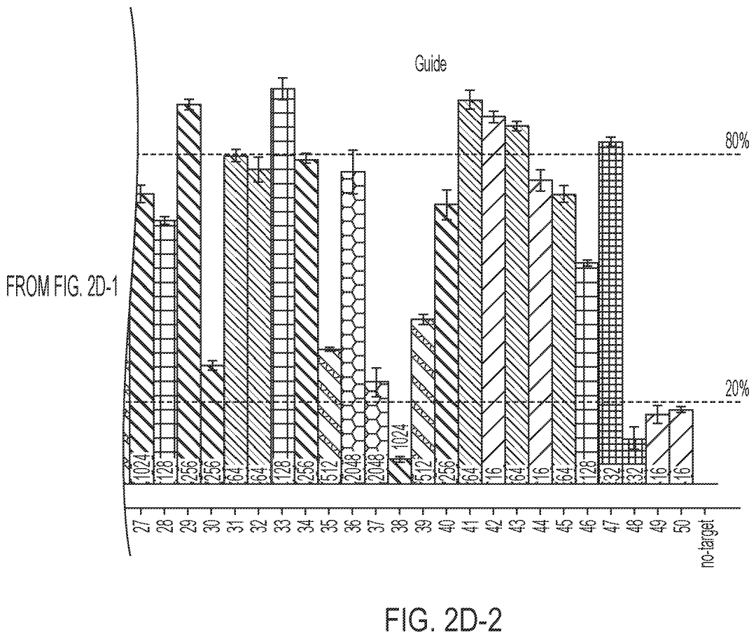

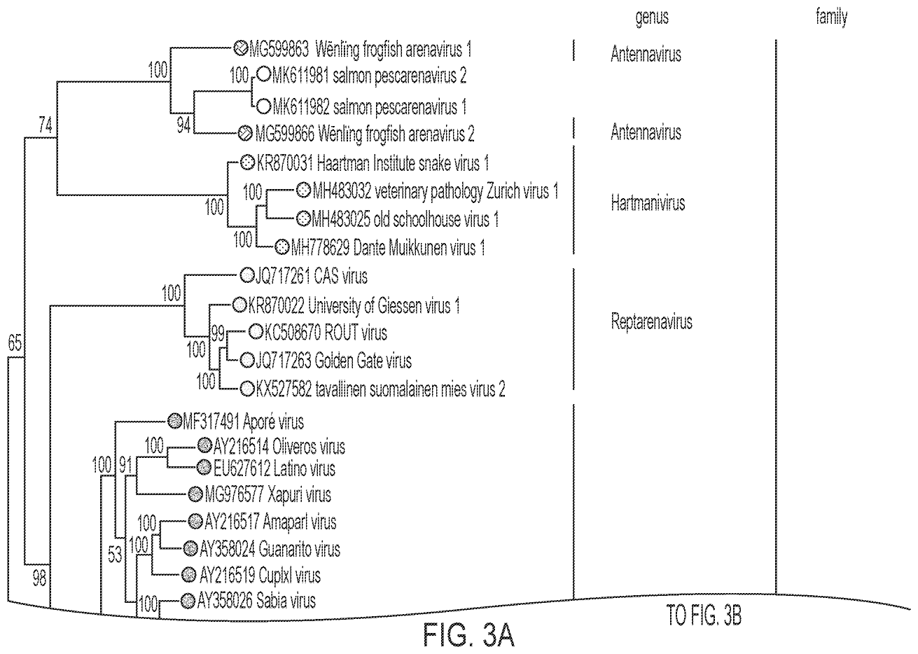

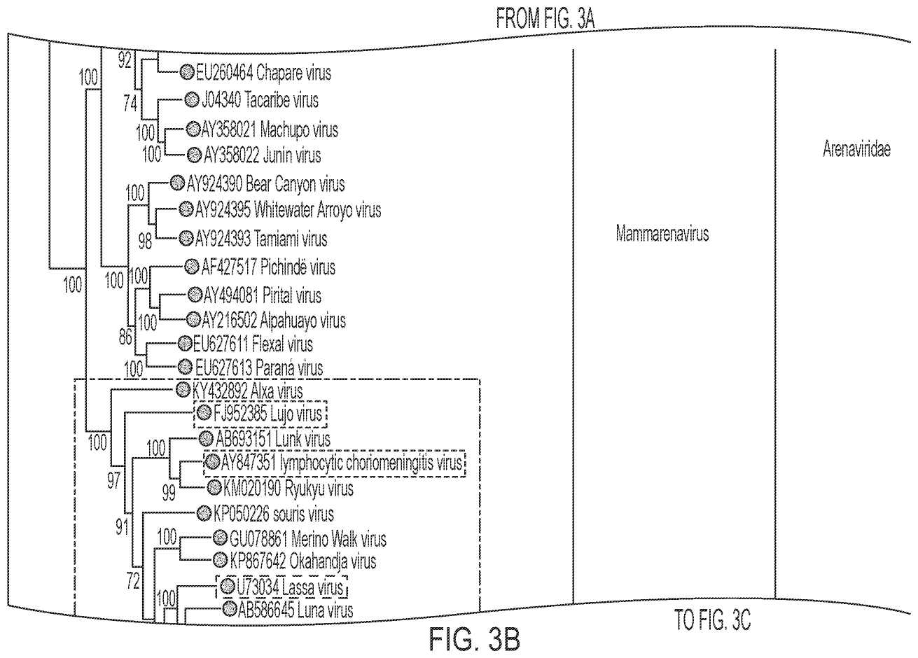

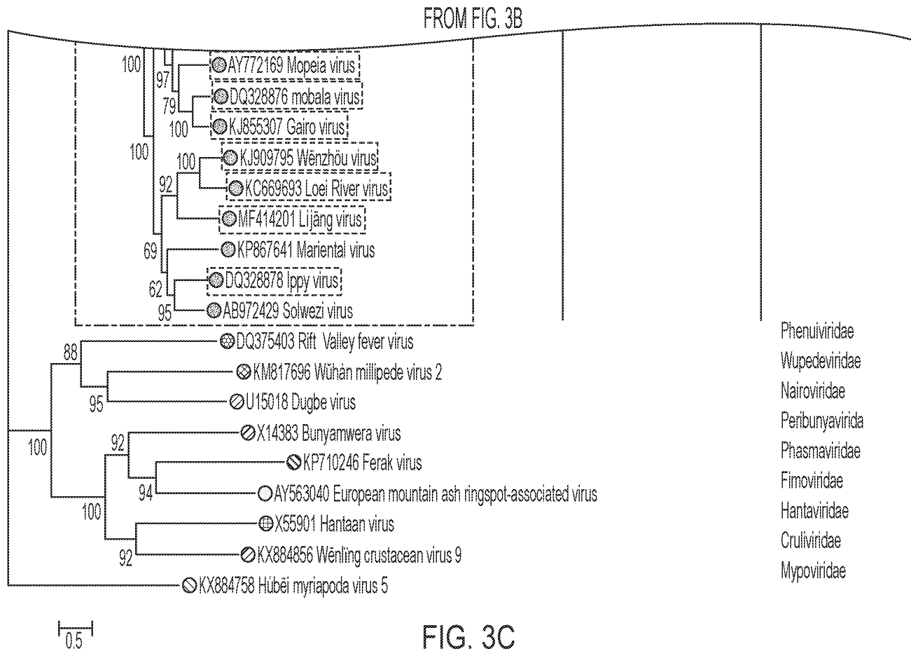

The performance testing results are shown in FIG. 2. Selected high performing crRNAs designed for L LASV target (#5, #9, #29 and #33, lineage II and IV versions for each of these crRNAs) were used to identify their performance against a panel of LASV targets representing all known lineages as well as near neighbor targets (representing closely related viral species) as seen in FIGS. 3A-3C.

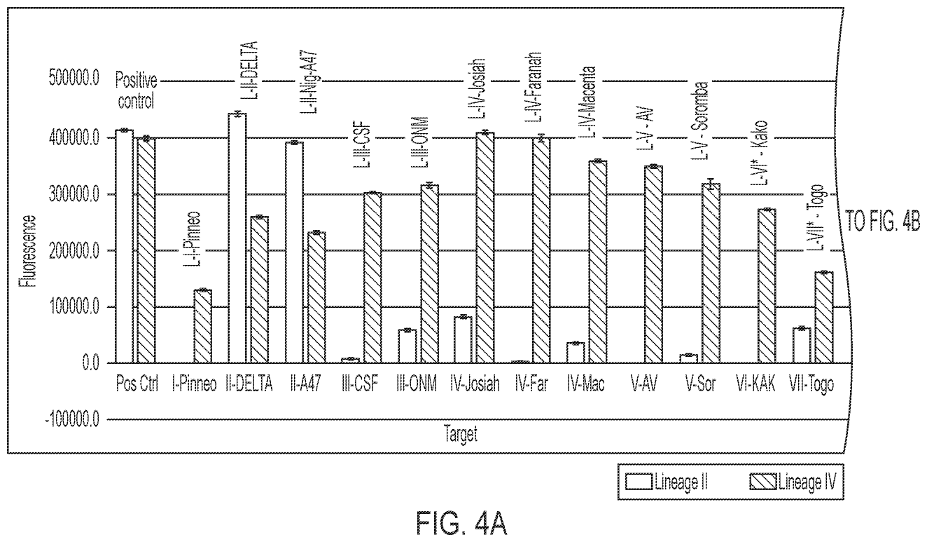

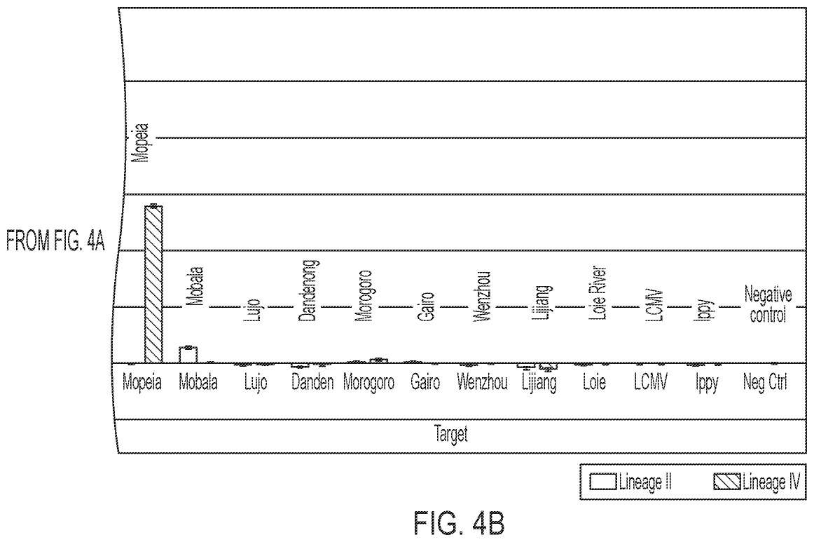

One of the crRNA molecules tested using this experimental setup (crRNA #5_LIV, SEQ ID No: 57) was able to detect all lineages of LASV while cross-reacting with only one near neighbor species (FIG. 4).

The data obtained in Cas13a assays using the eight crRNA sequences (#5, #9, #29 and #33, lineage II and IV versions for each of these crRNAs) was used to identify the variables affecting crRNA performance.

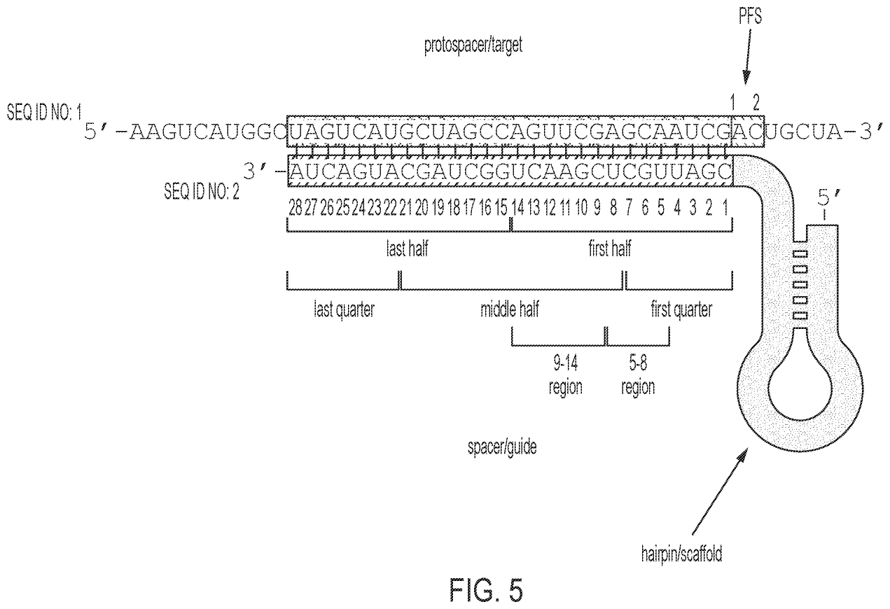

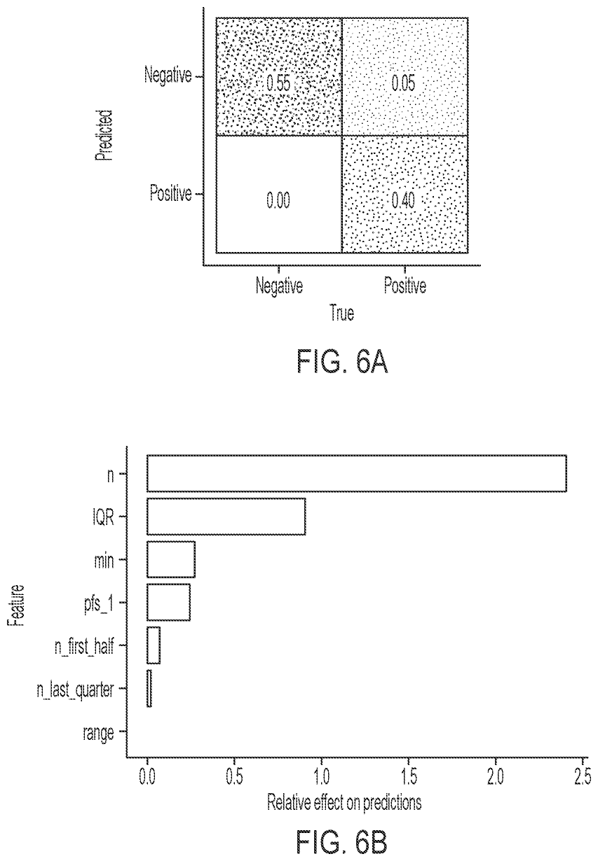

To understand how the distribution of mismatches and other variables influence the outcome of the detection assays, a RuleFit algorithm-based predictive model was developed using our experimental data. The RuleFit classifier was trained to predict whether a guide-target pair would yield a signal above or below threshold (defined as 20% of maximum signal) using the mismatch datasets. Following training, a variety of features were related to mismatch count and position (see FIG. 5 for location of the features in the crRNA). The output accuracy after 10-fold cross-validation was 95.0%±2.0% A representative confusion matrix of the predictions is shown in FIG. 6. The confusion matrix (FIG. 6A) compares the predictions of the model with the actual experimental data. The model shows more false negatives than false positives with an overall accuracy of 95%. Using this model, the rules were interrogated for feature importance (FIG. 6B). The feature with the overall highest global impact on the predictions was the total mismatch count (n). The analysis of the signals shows that for spacer/target pairings with the same number of mismatches can be significantly different indicating importance of other features for some mismatch ranges. The second most significant feature for all datasets was the interquartile range (IQR,) which reflects the distribution of mismatches across the spacer. IQR indicates whether the mismatches are clustered or are evenly distributed along the spacer sequence. The position of the 5′-most mismatch (min) and the identity of the nucleotide located at the protospacer flanking site #1 (PFS_1) also influence the prediction. The most important regions for mismatch count was the last quarter of the guide (or positions 21-28), first half of the guide (positions 1-14), and the region 5-8. Overall, the properties of these features suggest that, while overall mismatch count (n) was the most important, the location and distribution of those mismatches and identity of a nucleotide at PFS #1 had the strongest relative impact on classification (i.e. assay outcome).

The RuleFit classifier model identifies the prediction rules associating the most important features.

### Further Embodiments

While the example used the RuleFit machine learning model, other models could be used to develop suitable degenerate crRNAs.

Degenerate crRNA design features identified in this disclosure may be potentially used for targets other than LASV by selective grouping of the desired taxon sequences and exclusion of others using recommended feature ranges. A general algorithm for selection of degenerate crRNA candidates would be: (1) initial selection of crRNA sequences with degenerate spacers, (2) determination of mismatch positions between the spacer and all versions of the target and near-neighbor sequences, (3) use of the RuleFit classifier model to determine the Cas13a-based CRISPR assay for targets and near-neighbors and (4) assessment if the tested crRNA fulfils the desired criteria (detecting the intended targets and not cross-reacting with near-neighbors). If the criteria are not fulfilled, the algorithm generates new candidate crRNA and goes to step 2, otherwise step 5, which entails in vitro validation of the optimized crRNAs.

Suitable Cas-based assay systems include those known in the art such as those described in Spangler, J. R., Leski, T. A., Schultzhaus, Z. et al. “Large scale screening of CRISPR guide RNAs using an optimized high throughput robotics system,” which is incorporated herein by reference for the purposes of teaching Cas-based assays.

### Advantages

The design of degenerate crRNAs for detection of highly variable nucleic acid targets as described herein offers the following advantages over alternatives. First, the use of a single degenerate crRNA for detection of variable targets avoiding multiplexing of CRISPR based detection reactions for detection of broad taxonomic ranges, with degenerate crRNA composed of up to 2048 guide sequence permutations were shown to perform well. Second, the technique offers the potential for rapid design of crRNA sequences for any variable target using RuleFit based design rules. In contrast, most current design methods require extensive empirical testing of designed crRNAs in vitro while those existing automated design methods (e.g. ADAPT) do not allow using degenerate crRNAs and require multiple crRNAs to detect highly variable targets. Finally, this technique makes it easy to synthesize and test degenerate RNAs, whereas alternative methods use expensive and inflexible direct RNA synthesis with modified bases.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

Detection of Pathogens (Including LASV) with Highly Variable Sequences
Demby, A. H., Chamberlain, J., Brown, D. W. & Clegg, C. S. Early diagnosis of Lassa fever by reverse transcription-PCR. J Clin Microbiol 32, 2898-2903 (1994).
Kreer, C. et al. openPrimeR for multiplex amplification of highly diverse templates. J Immunol. Methods 480, 112752, doi:10.1016/j.jim.2020.112752 (2020).
Krysler, A. R., Cromwell, C. R., Tu, T. et al. Guide RNAs containing universal bases enable Cas9/Cas12a recognition of polymorphic sequences. Nat Commun 13, 1617 (2022). https://doi.org/10.1038/s41467-022-29202-X.
Li, K., Shrivastava, S. & Stockwell, T. B. Degenerate primer design for highly variable genomes. Methods Mol Biol 1275, 103-115, doi:10.1007/978-1-4939-2365-6_7 (2015).
McCormick, J. B., Webb, P. A., Krebs, J. W., Johnson, K. M. & Smith, E. S. A prospective study of the epidemiology and ecology of Lassa fever. J Infect Dis 155, 437-444 (1987).
Bowen, M. D. et al. Genetic diversity among Lassa virus strains. J Virol 74, 6992-7004 (2000).

### CRISPR/Cas Biosensing

Abudayyeh, O. O., et al. (2017). “RNA targeting with CRISPR-Cas13.” Nature 550(7675): 280-284.
Abudayyeh, O. O., et al. (2016). “C2c2 is a single-component programmable RNA-guided RNAtargeting CRISPR effector.” Science 353(6299): aaf5573.
Ackerman, C. M., et al. (2020). “Massively multiplexed nucleic acid detection with Cas13.” Nature 582(7811): 277-282.
Barnes, K. G., et al. (2020). “Deployable CRISPR-Cas13a diagnostic tools to detect and report Ebola and Lassa virus cases in real-time.” Nature communications 11(1): 1-10.
Kellner, M. J., et al. (2019). “SHERLOCK: nucleic acid detection with CRISPR nucleases.” Nature protocols 14(10): 2986-3012.
Metsky, H. C., et al. (2020). “CRISPR-based surveillance for COVID-19 using genomicallycomprehensive machine learning design.” BioRxiv.
Myhrvold, C., et al. (2018). “Field-deployable viral diagnostics using CRISPR-Cas13.” Science 360(6387): 444-448.
Sohail, M., et al. (2022). “Molecular reporters for CRISPR/Cas: From design principles to engineering for bioanalytical and diagnostic applications.” TrAC Trends in Analytical Chemistry 149: 116539.

### Algorithmic Approaches

Friedman, J. H. & Popescu, B. A. Predictive learning via rule ensembles. Annals of Applied Statistics 2, 916-954 (2008).
Metsky, H. C. et al. Designing sensitive viral diagnostics with machine learning. Nat Biotechnol 40, 1123-1131, doi:10.1038/s41587-022-01213-5 (2022).

What is claimed is:

1. A method for detecting members of a phylogenetically diverse group, the method comprising:
(1) identifying conserved regions in a set of diverse nucleic acids in the phylogenetically diverse group;
(2) designing candidate degenerate complementary spacer regions of CRISPR guide RNAs (crRNAs) corresponding to the conserved regions;
(3) conducting high-throughput screening of the candidate degenerate crRNAs against complementary synthetic targets to obtain high performing degenerate crRNAs;
(4) conducting high-throughput screening of the high performing degenerate crRNAs against targets representing at least a majority of the phylogenetically diverse group and at least one target representing a near neighbor to the phylogenetically diverse group to obtain a dataset; and
(5) using a machine learning algorithm to analyze the dataset to identify generalizable crRNA design rules for detection of members of the phylogenetically diverse group.

2. The method of claim 1, wherein the targets representing at least a majority of the phylogenetically diverse group represent all known members of the group.

3. The method of claim 1, wherein the phylogenetically diverse group is a pathogen.

4. The method of claim 3, wherein the pathogen is a virus.

5. The method of claim 4, wherein the virus is Lassa virus.

6. The method of claim 1, wherein the machine learning algorithm is RuleFit.

7. The method of claim 3, further comprising:
(6) using said generalizable crRNA design rules to produce a working set of degenerate crRNAs and using these to detect the presence or absence of the pathogen in at least one sample.

8. The method of claim 7, wherein said at least one sample comes from one or more patients, and further comprising:
(7) using the working set of degenerate crRNAs to identify samples comprising said pathogen and administering suitable treatment to the one or more patients associated with the identified samples.

9. A CRISPR guide RNA comprising a nucleic acid sequence selected from the group consisting of SEQ ID NOs: 3 through 141, inclusive.

10. The CRISPR guide RNA of claim 9, wherein said nucleic acid sequence is SEQ ID NO: 57.

11. A Cas-based assay system comprising CRISPR guide RNA comprising a nucleic acid sequence selected from the group consisting of SEQ ID NOs: 3 through 141, inclusive.

12. The Cas-based assay system of claim 11, wherein said nucleic acid sequence is SEQ ID NO: 57.
